(19) United States
(12) Patent Application Publication
(10) Pub. No.: US 2014/0312054 A1
(43) Pub. Date: Oct. 23, 2014

(54) DISPENSER FOR ELECTROCHEMICAL SENSORS

(71) Applicant: LifeScan Scotland Limited, Inverness-shire (GB)

(72) Inventor: John Crawford, Inverness (GB)

(21) Appl. No.: 14/002,969

(22) PCT Filed: Dec. 13, 2012
(86) PCT No.: PCT/EP12/75454

§ 371 Date: Sep. 3, 2013

(51) Int. Cl. B65D 83/04; G01N 33/487; B65D 83/08

(52) U.S. Cl. CPC B65D 83/0418; B65D 83/0829; G01N 33/48757; G01N 33/48778
USPC 221/232

(57) ABSTRACT

The invention provides dispensers for sensors. The dispensers of the invention are capable of storing a plurality of sensors and dispensing them one-by-one.

### FIELD OF THE INVENTION

The present invention relates to dispensers for electrochemical sensors. In particular, the invention relates to dispensers suitable for dispensing small sensors for use.

### BACKGROUND OF THE INVENTION

Methods and devices for the analyte detection and concentration measurement in a fluid sample are well known. For example, various devices and methods are known for determining glucose, ketone bodies, cholesterol, lipoproteins, triglycerides, acetaminophen or HbAlc concentrations in a sample of a bodily fluid such as urine, blood, plasma or interstitial fluid. Such determinations can be achieved using analytical sensors such as test strips, based on, for example, visual, photometric or electrochemical techniques.

In an electrochemical technique, a fluid sample is placed into a sample chamber of an electrochemical cell of a sensor that includes at least a counter and working electrode. The analyte reacts with a redox reagent in the cell to form an oxidizable or reducible substance. The quantity of oxidizable or reducible substance is electrochemically estimated and related to the amount of the analyte present in the sample.

Sensors for use in analyte testing, in which sensors electrochemical cells are incorporated, typically use a carrier material to provide structural integrity, facilitate handling, and establish an electrical connection between the cell to a meter. The carrier materials may take any form, but typically are in the form of a test strip. The costs of the sensors are related to the materials used in the electrochemical cell. For sensors, or strips, in which a large portion of the device is composed of the cell, it is desirable to keep the size of the sensor small to reduce costs. However, users of these small sensors find them difficult to handle. Additionally, a small-sized strip or sensor increases the potential for contaminating the port of the meter, into which the sensor is placed, with the fluid being analyzed. Therefore, it is desirable to provide a dispenser capable of dispensing small sensors that avoids these disadvantages.

### BRIEF DESCRIPTION OF THE DRAWINGS

FIG. 1 is a top perspective view of a dispenser of the invention.

FIG. 1A is a magnified view of the top of the dispenser shown in FIG. 1.

FIG. 2 is a side view of the dispenser of FIG. 1 with the lid open.

FIG. 3 is a side view of the dispenser of FIG. 1 with lid closed.

FIG. 4 is a front view of the dispenser of FIG. 3 with the lid closed

FIG. 5 is a back view of the dispenser of FIG. 3 with the lid closed

FIG. 6 is a front view of the dispenser of FIG. 2 with the lid open.

FIG. 7 is a cross-sectional view of the dispenser of FIG. 6 through line I-I.

FIG. 7A is a magnified view of the top of the dispenser shown in FIG. 7.

FIG. 8 is an exploded view of the device of FIG. 5.

FIG. 8A is a magnified view of the top surface of the device of FIG. 8.

FIG. 9 is a cross-sectional view of the dispenser of FIG. 4 through line II-II.

FIG. 9A is a magnified view of a portion of the dispenser of FIG. 9.

FIG. 10 is a top perspective view of an the outer housing for a sensor cassette useful in the dispenser of the invention.

FIG. 10A is magnified, top perspective view of the outer housing for a sensor cassette useful in the dispenser of the invention.

FIG. 11 is magnified, top perspective view of a strip cassette useful in the dispenser of the invention.

FIG. 12A is a magnified, top perspective view of the lid of a dispenser to the invention prior to deployment of the pusher.

FIG. 12B is a magnified, top perspective view of the lid of a dispenser to the invention after deployment of the pusher.

FIG. 13A is a cross-section view of a portion of the dispenser with the lid closed.

FIG. 13B is a cross-section view of a portion of the dispenser with the lid partially opened.

FIGS. 13C is a cross-section view of a portion of the dispenser with the lid fully opened.

### DETAILED DESCRIPTION OF THE INVENTION

In general, the invention provides dispensers for electrochemical-based and photometric sensors. The dispensers of the invention likely will find greatest utility in the dispensing of small sensors, such as electrochemical modules (“ECMs”), test strips and the like, useful for the determination of an analyte in a bodily fluid sample. The dispensers of the invention are capable of storing a plurality of sensors and dispensing them one-by-one.

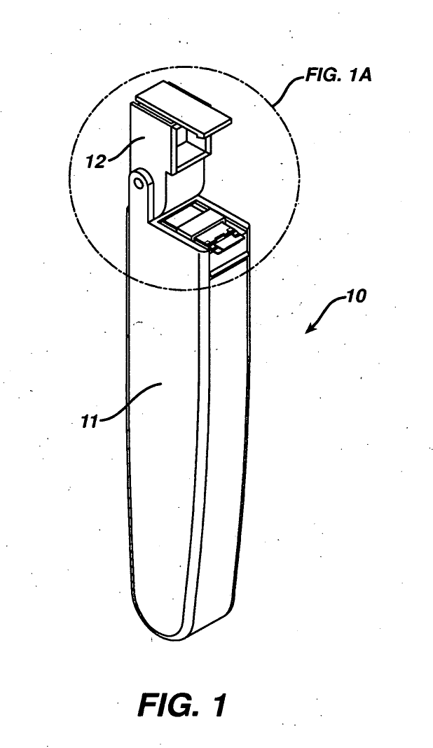

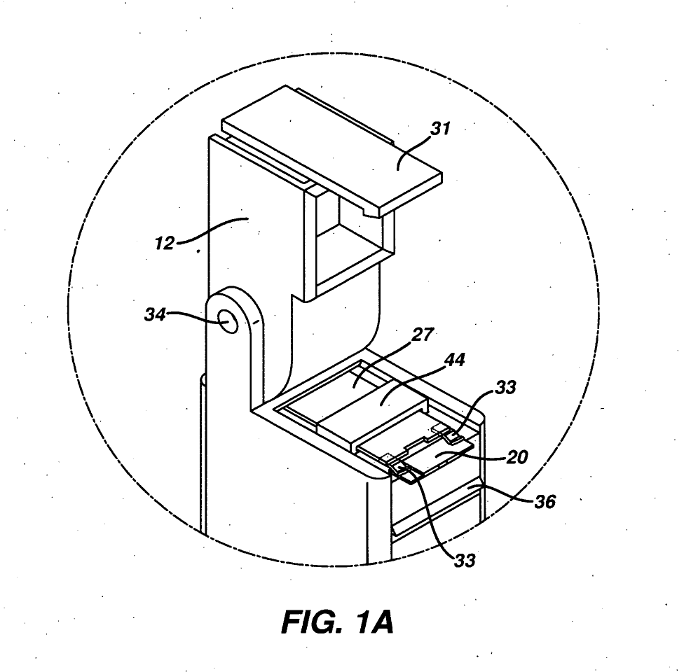

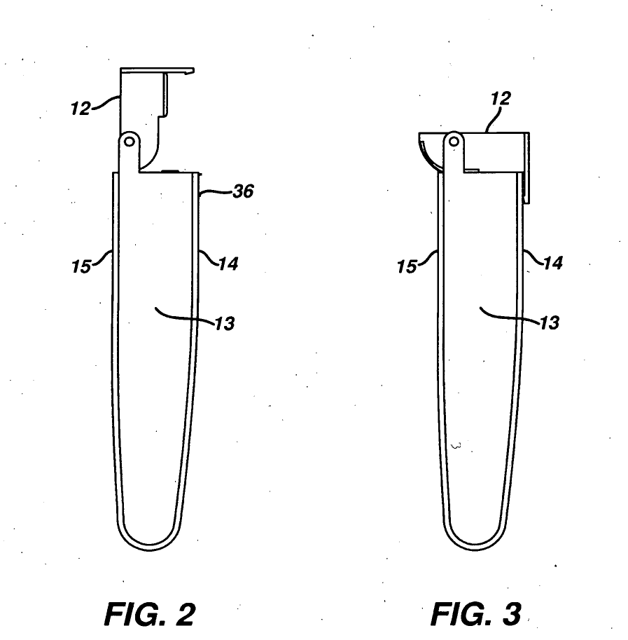

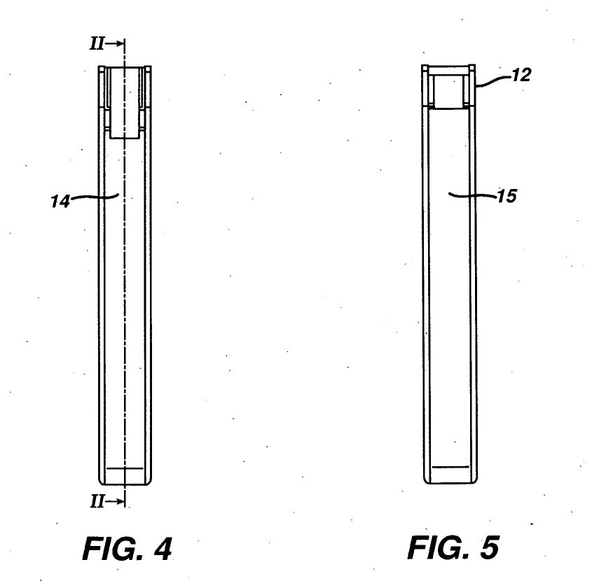

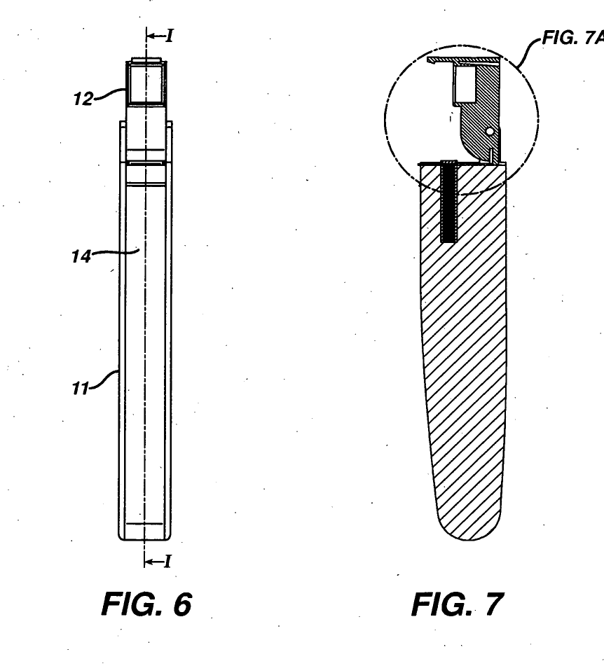

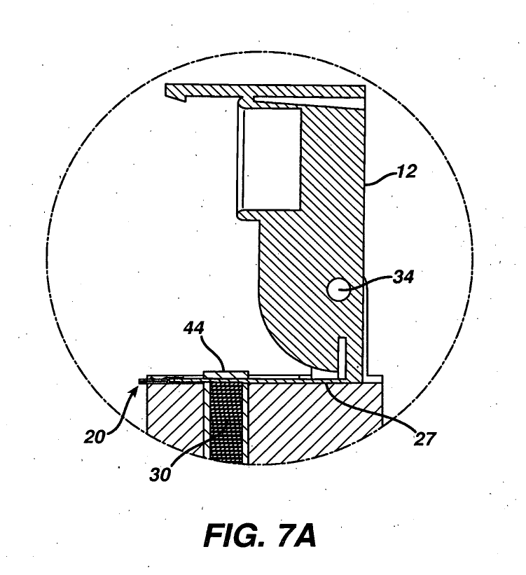

In FIG. 1 is shown a dispenser 10 of the invention with housing 11 and lid 12 atop housing 11. In FIG. 2 is depicted a side view of dispenser 10 with lid 12 open and in FIG. 3 is a side view of the opposite side of dispenser 10 with lid 12 closed, in which side surfaces 13, front surface 14 and back surface 15 of housing 11 are shown. The overall shape of the dispenser 10 as shown is rectangular in shape with a rounded, tapered bottom portion, but the dispenser may be of any convenient size and shape. Preferably, the dispenser is sized to be suitable to hold and dispense the number of sensors desired and more preferably, is sufficiently small so as to fit within the palm of a hand. The housing may be formed of any suitable material and preferably is substantially impermeable to moisture and gases and is sufficiently hard so that the housing is lion-deformable. Suitable materials include thermoplastic materials including, without limitation, acrylonitrile butadiene styrene, polycarbonate, polyethylene terphthalate. Preferably the housing is made of polyethylene terphthalate. Also, preferably the housing is produced as a single piece using any known manufacturing process including without limitation, injection, cast or other suitable molding method.

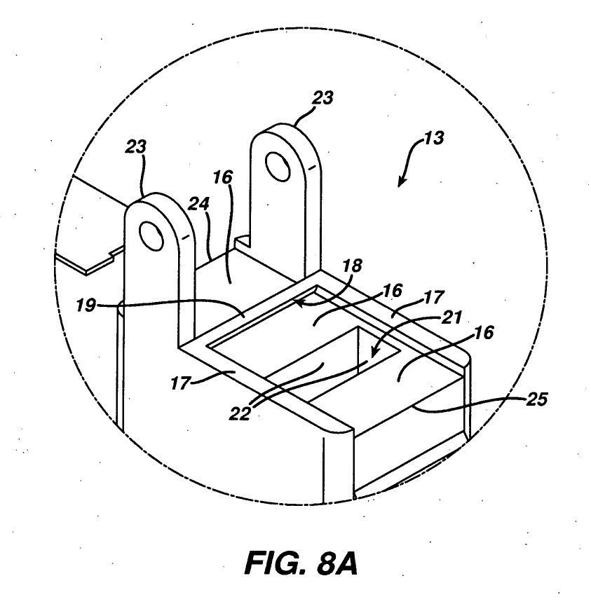

The top of housing 11, as shown in FIG. 8A, includes top surface 16, which surface is recessed between edges 17 that extended upwardly from the side walls 13 of housing 11. Additionally, preferably top surface 16 has pusher opening 18 formed within ridge 19 that extends between edges 17. Also shown are hinges 23 that are coextensive with and extend upwardly from side walls 13 of housing 11. The hinges, as shown, are inset a short distance from the back edge 24 of top surface 16, but need not be. Additionally, hinges 23 are inset a longer distance from front edge 25 to allow for full extension of pusher 27 of lid 12 and to provide support for the sensor or strip being dispensed.

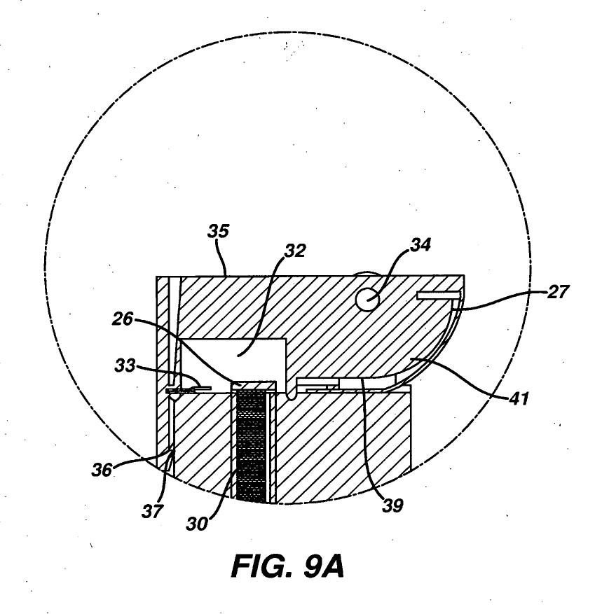

A lid is movably attached to housing 11. As shown in FIG. 1A, lid 12 is attached via hinges to hinges 23 via pivots (not shown) that insert into pivot holes 34. Pusher 27 extends outwardly from lid 12 when lid 12 is in the open position and passes through opening 18 of the dispenser top surface. Additionally, pusher 27 extends through slot 43, most easily seen in FIG. 10A in outer housing 26 of sensor cassette 30. As shown in FIG. 1A, pusher 27 is in its fully extended position having pushed a sensor 20 out of slot 43 of outer housing 26 and under contacts 33 that are fixedly attached to top surface 16 of housing 11.

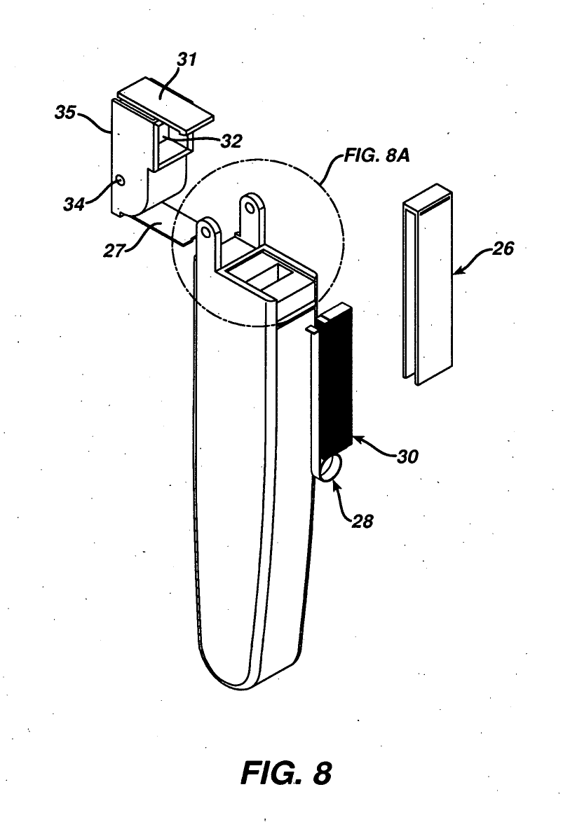

With reference to FIG. 8A, aperture 21 in top surface 16 provides access to a chamber within housing 11, the chamber being formed by four internal walls 22, only two of which are shown in FIG. 8A. The internal walls preferably have a dessicant incorporated therein. The chamber within housing 11 is suitably sized and shaped to receive cassette outer housing 26 containing cassette 30 of a plurality of sensors to be dispensed. When cassette outer housing 26 resides within the chamber of the housing, the top-most portion of the cassette outer housing extends above top surface 16 of housing 11 so that slot 43 in the outer housing is even with top surface 16 so as to permit exit of a sensor as described herein below. The chamber loaded with the outer housing and a cassette is shown in the cross-section view in FIG. 7. Aperture 21 in top surface 16 of housing 11 allows for loading the cassette and outer housing into, and removal out of, the device housing 11.

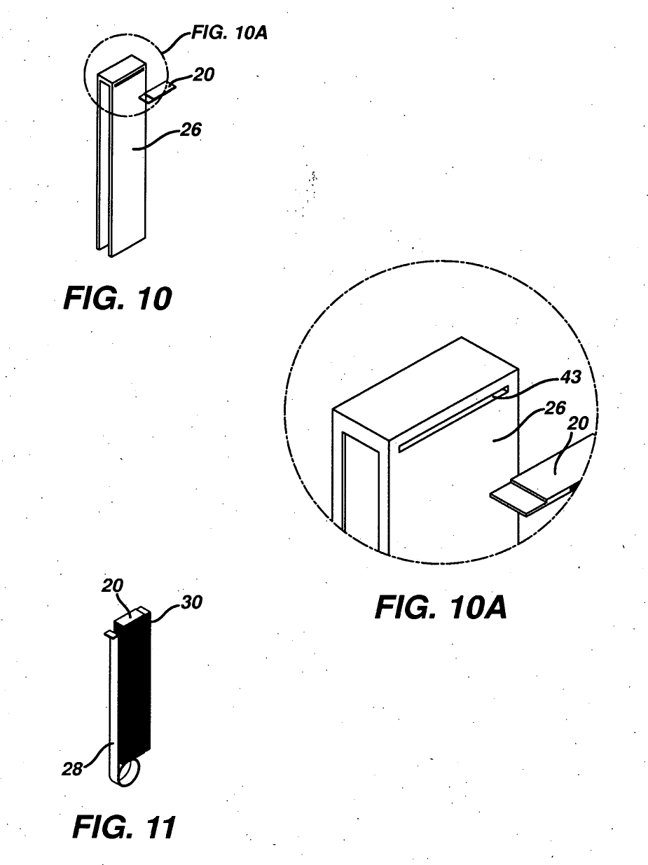

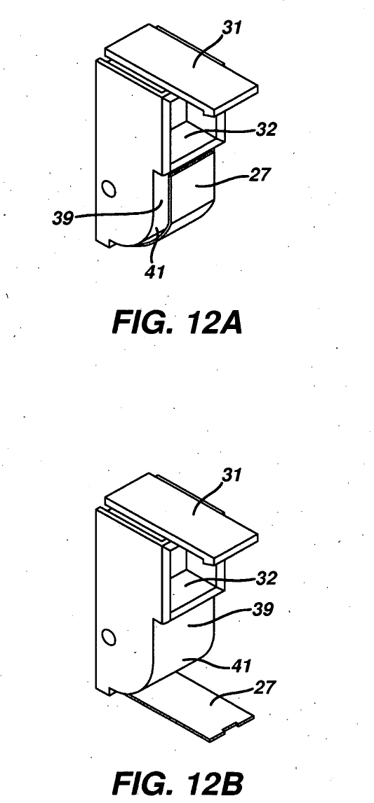

An exploded view of the cassette and outer housing are shown in FIG. 8 and the outer housing and cassette are individually shown in FIGS. 10 and 11, respectively. A biasing element 28 biases the cassette of sensors 30 upwardly toward the top of cassette outer housing 26 such that when one sensor is dispensed, another is provided ready to be dispensed. Biasing element 28 may be any suitable resilient element including, without limitation, a coiled or semi-coiled spring, a pneumatic plunger, a worm drive with a coiled spring, an extension spring, a tension spring and the like.

Each of the stored sensors is stored so that it is in a ready-to-use orientation when dispensed. For example, the sensors may be oriented to make contact with the strip port connector of a meter or other device and in an orientation such that blood or other fluid to be analyzed may be applied once the sensor is inserted into the meter or other device.

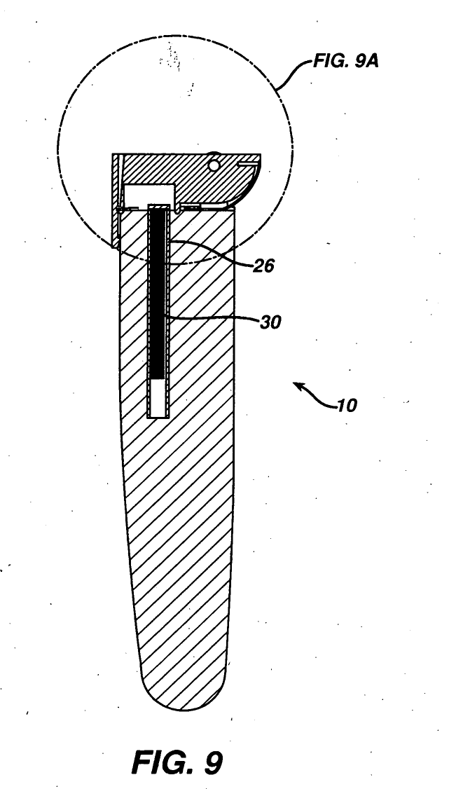

Lid 12 is hingedly affixed to hinges 23 as shown, for example, in FIGS. 1A, 2 and 7, at pivot hole 34. A front tab 31 extends downwardly from lid top surface 35 and overlaps the uppermost portion of front surface 14 of dispenser 11. As shown in FIG. 9A, a protrusion 37 extends inwardly from inner surface of front tab 31 and releasably engages with ledge 36 that extends outwardly from dispenser front surface 14 to latch lid 12 closed. Lifting of front tab 31 away from dispenser front surface 14 releases the engagement and permits opening of lid 12. One ordinarily skilled in the art will recognize that any suitable closing mechanism may be used for latching lid 12.

Lid 12 additionally includes sealing chamber 32. When lid 12 is in the closed position, chamber 32 will overlay top surface 16 fitting tightly into the recess formed between edges 17 and tightly abutting ridge 17. Preferably, the chamber is composed of a material having a low gas permeability, a Shore-A hardness of about 55 to 85, and abrasion resistance. Suitable materials include SANTOPRENE™, polytetrafluoroethylene, and the like. Chamber 32 preferably is sufficiently large so as to cover the dispenser top surface 16 between edges 17 as well as the top surface of cassette outer housing 26 and contacts 33.

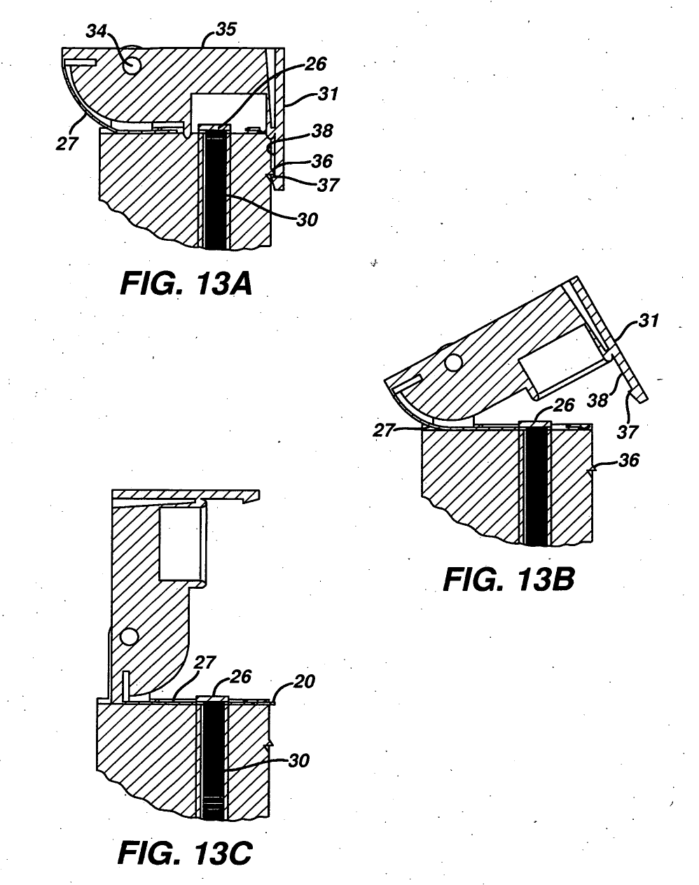

Lid 12 may be of any suitable size and shape, but as shown rear portion 33 of lid 12 is composed of a front-most flat portion 39 that is coextensive with curved rearmost portion 41. As shown on FIGS. 13A-C, pusher 27 is movably affixed to Curved portion 41 so that, when lid 12 is raised upwardly away from dispenser top surface 16, pusher 27 uncurls and moves forwardly along top surface 16. As pusher 27 advances, it passes through opening 18 in ridge 19 and engages a sensor that is held at the top of cassette outer housing 26. As pusher 27 continues its advancement, the sensor is moved out of slot 43 in cassette outer housing 26 onto surface 16 of housing 11. With the continued advancement of pusher 27 along surface 16, the sensor is pushed underneath contacts 33 as pusher 27 continues its forward movement. Preferably, one or more contacts are provided on top surface 16 of housing 11. At full extension of pusher 27, the sensor is partially underneath contacts 33 and partially extended past edge 25 of housing 11. Once the sensor is removed from housing 11, the lid is rotated downwardly retracting pusher 27 into its retracted position. Pusher 27 may be formed from any material that is sufficiently flexible to curl and uncurl as required in the use of the device, but which is sufficiently hard to enable the pusher to engage and push a sensor forward from the outer housing 26.

What is claimed:

1. A device for dispensing sensors, comprising:
a. a housing comprising a top surface and a chamber within the housing, wherein the chamber is in communication with the top surface;
b. an outer cassette housing removably held within the chamber, the outer cassette housing comprising a first surface, a second surface, and a slot through the second surface for movement of a sensor out of the outer cassette housing;
c. a cassette containing a plurality of sensors within the outer cassette housing, wherein the cassette comprises a biasing element for biasing the cassette toward the first surface of the outer housing; and
d. a lid movably attached to the housing for movement between a closed and an open position, the lid comprising a pusher attached thereto capable of moving a sensor out of the slot in the outer cassette housing when the lid is in the open position.

2. The dispenser of claim 1, wherein the top surface of the housing further comprises one or more contacts thereon.
